(19) United States
(12) Patent Application Publication
(10) Pub. No.: US 2017/0325798 A1
(43) Pub. Date: Nov. 16, 2017

(54) WOUND RETRACTOR SPECIMEN BAG

(71) Applicant: Covidien LP, Mansfield, MA (US)

(72) Inventor: Scott Prior, Shelton, CT (US)

(21) Appl. No.: 15/467,392

(22) Filed: Mar. 23, 2017

Related U.S. Application Data
(60) Provisional application No. 62/336,047, filed on May 13, 2016.

(51) Int. Cl. A61B 17/02

(52) U.S. Cl. CPC A61B 17/0218; A61B 17/0293

(57) ABSTRACT

A surgical apparatus for positioning within a tissue incision to access an underlying body cavity and remove a tissue specimen therefrom includes a wound retractor having a specimen bag attached thereto. The wound retractor includes a proximal ring, a distal ring, and a film extending therebetween defining a longitudinal passage therethrough. The specimen bag is attached to the distal ring of the wound retractor and permits placement of a tissue specimen to be removed from the body therein.

### CROSS-REFERENCE TO RELATED APPLICATIONS

This application claims the benefit of and priority to U.S. Provisional Patent Application No. 62/336,047 filed May 13, 2016, the entire disclosure of which is incorporated by reference herein.

### TECHNICAL FIELD

The present disclosure relates generally to surgical apparatuses for use in minimally invasive surgical procedures, such as endoscopic and/or laparoscopic procedures, and more particularly, the present disclosure relates to a surgical apparatus including both a wound protection device and a specimen retrieval bag for collecting body tissue(s) and/or body fluid(s) during these procedures.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of the body cavity. The surgeon can then perform diagnostic and/or therapeutic procedures at the surgical site with the aid of specialized instrumentation, such as forceps, graspers, cutters, applicators, and the like, which are designed to fit through additional cannulas. To protect the opening from accidental penetration by the surgical instruments, wound retractors and wound protectors are often placed across the opening.

When removing certain tissue from the body cavity, for example tumor tissue, it is important that the tumor tissue does not come into contact with healthy or uninvolved tissue, so as in this way to avoid metastasis and avoid harming the patient. Minimally invasive surgical procedures, however, may be limited where large size tumors or large masses of tissue have to be removed from a body cavity. If tumor tissue or tissue parts have to be removed, they may be introduced into an “extraction bag,” also referred to herein as a “specimen bag,” at the site where the tumor or diseased tissue has been detached from the surrounding tissue, after which the specimen bag is withdrawn from the body, normally through a trocar or similar device, thereby minimizing contact of the diseased tissue with healthy tissue.

In some instances, depending upon the volume of tissue being removed from the body, the tissue within the specimen bag must be broken up prior to removal from the body to allow for the specimen bag and its contents to pass through the opening used to conduct the endoscopic procedure.

Improved wound retractors and specimen bags for use in minimally invasive surgical procedures remain desirable.

### SUMMARY

The present disclosure is directed to surgical apparatuses and kits for use in minimally invasive surgery. The surgical apparatus includes a wound retractor having a specimen bag attached thereto, for placement of diseased tissue therein. In embodiments, the kit of the present disclosure includes the surgical apparatus, a morcellating trocar for breaking up tissue prior to its removal from the body, and an optional cannula.

A surgical apparatus of the present disclosure includes, in embodiments, a wound retractor including a proximal ring, a distal ring, and a film disposed between the proximal ring and the distal ring. The surgical apparatus also includes a specimen bag attached to the distal ring of the wound retractor.

In embodiments, the proximal ring of the wound retractor defines a generally circular opening and is deformable. Similarly, the distal ring of the wound retractor may also define a generally circular opening and is deformable.

In some embodiments, the film of the wound retractor defines an adjustable length between the proximal ring and the distal ring.

In certain embodiments, the specimen bag is attached to the distal ring of the wound retractor by a method such as adhesive bonding, welding, heat-sealing, and combinations thereof.

In embodiments, the proximal ring, the distal ring, or both, possess a tether thereon.

A kit on the present disclosure includes, in embodiments, a surgical apparatus including a wound retractor having a proximal ring, a distal ring, a film disposed between the proximal ring and the distal ring, and a specimen bag attached to the distal ring of the wound retractor. The kit of the present disclosure also includes a morcellating trocar and an optional cannula.

In embodiments, the morcellating trocar includes a proximal portion, a distal portion, and an elongate shaft between the proximal portion and the distal portion, the elongate shaft having blades on an interior surface at the distal portion projecting into a lumen of the elongate shaft, and the proximal portion of the elongate shaft possessing a rotation knob for rotating the elongate shaft.

Methods of the present disclosure include, in embodiments, introducing into a body opening a surgical apparatus including a wound retractor having a proximal ring, a distal ring, a film disposed between the proximal ring and the distal ring, and a specimen bag attached to the distal ring of the wound retractor, by inserting the surgical apparatus through the body opening into the body cavity. A tissue specimen is then passed through the proximal ring, the film and the distal ring of the surgical apparatus into the specimen bag, after which the proximal ring is removed from the body cavity through the body opening so that the film is adjacent tissue encompassing the body opening. The proximal ring is then pulled away from the distal ring such that the distal ring is brought into abutment with an inner surface of the tissue surrounding the body opening, and the proximal ring is rolled about itself such that the film is furled about the proximal ring and tensioned. The proximal ring is further rolled to retract the incision and bring the proximal ring into abutment with an outer surface of the tissue. The tissue specimen is then removed from the specimen bag.

In embodiments, the methods of the present disclosure further including breaking up the tissue specimen prior to removing the tissue specimen from the specimen bag.

In certain embodiments, the methods of the present disclosure further include introducing a cannula through the proximal ring, the film, and the distal ring into the specimen bag prior to removing the tissue specimen from the specimen bag. A morcellating trocar is then introduced through the cannula into the specimen bag, followed by introduction of a grasping element through the morcellating trocar into the specimen bag. The grasping element grasps the tissue specimen therein, and the grasping element is then pulled proximally while activating the morcellating trocar to break up the tissue specimen as it is removed from the specimen bag.

In some embodiments, the cannula possesses an anchoring balloon which is inflated to anchor the cannula in the body opening. In certain embodiments, the specimen bag is also inflated prior to removal of the tissue specimen therefrom.

In embodiments, the morcellating trocar includes a proximal portion, a distal portion, and an elongate shaft between the proximal portion and the distal portion, the elongate shaft having blades on an interior surface at the distal portion projecting into a lumen of the elongate shaft, and the proximal portion of the elongate shaft possessing a rotation knob for rotating the elongate shaft. Activating the morcellating trocar includes rotating the elongate shaft so that the tissue specimen is cut by the blades as the tissue specimen passes through the morcellating trocar.

### BRIEF DESCRIPTION OF THE DRAWINGS

Various embodiments are described herein with reference to the drawings wherein:

FIG. 1 is a perspective view of the components of a kit of the present disclosure;

FIG. 2 is a perspective view of a surgical apparatus of the present disclosure, including a specimen bag affixed to a wound retractor;

FIG. 3 is a perspective view of a morcellating trocar of the present disclosure;

FIG. 4 is an enlarged view of the indicated area of detail shown in FIG. 3;

FIG. 5 is a cross-sectional view of the distal end of the morcellating trocar in accordance with the embodiment of FIG. 3;

FIG. 6 is a perspective view of a patient's body, showing a wound, incision, and tissue specimen to be removed 320 from the body cavity;

FIG. 6A is a perspective view showing the partial removal of a surgical apparatus of the present disclosure through an incision into a body cavity;

FIG. 6B is a perspective view of a surgical apparatus of the present disclosure in a body cavity, adjacent tissue to be removed from the body cavity;

FIG. 7 is a perspective view of a surgical apparatus in accordance with the embodiment of FIG. 2, showing the wound retractor inserted into an incision in tissue and the specimen bag within the body cavity;

FIG. 8 is a perspective view of a surgical apparatus in accordance with the embodiment of FIG. 7, showing a cannula inserted through the wound retractor into the specimen bag;

FIG. 9 is a perspective view of a surgical apparatus in accordance with FIG. 8, showing a morcellating trocar of the present disclosure inserted through the cannula into the specimen bag;

FIG. 10 is a perspective view of a grasping element deployed through the morcellating trocar in accordance with the embodiment of FIG. 9, beginning to remove tissue from the specimen bag; and

FIG. 11 is a perspective view of a grasping element removing tissue through the morcellating trocar in accordance with the embodiment of FIGS. 9-10.

### DETAILED DESCRIPTION

The present disclosure provides a surgical apparatus for use in minimally invasive procedures. As used herein with reference to the present disclosure, minimally invasive surgical procedures encompass laparoscopic procedures and endoscopic procedures, and refer to procedures utilizing scopes or similar devices having relatively narrow operating portions or a small incision in the skin.

The aspects of the present disclosure may be modified for use with various methods for retrieving tissue specimens during minimally invasive procedures. Examples of minimally invasive procedures include, for example, cholecystectomies, appendectomies, nephrectomies, colectomies, splenectomies, and the like.

As used herein, the term distal refers to the portion of a surgical apparatus, including a wound retractor and specimen bag of the present disclosure, which is farthest from the user, while the term proximal refers to that portion of the surgical apparatus of the present disclosure which is closest to the user.

The surgical apparatus of the present disclosure includes a wound retractor with a specimen bag affixed thereto. The wound retractor component includes a sleeve member possessing at least two rings, including a proximal ring and a distal ring with a film between the two rings. The specimen bag component is attached to the distal ring of the wound retractor. In use, the entire surgical apparatus is passed through an incision and placed within the body, and tissue to be removed therefrom, referred to in embodiments as a “tissue specimen”, is passed through the wound retractor component and placed within the specimen bag. The proximal ring of the wound retractor component is then removed from the body cavity and placed adjacent the skin on the outside of the body adjacent the incision, with the distal ring of the sleeve member and specimen bag remaining within the body, so that the film extends through the incision and is adjacent the tissue encompassing the incision into the body. These apparatuses are suitable for use in any procedure where access to the interior of the body is limited to one or more relatively small incisions, as in minimally invasive procedures.

Kits of the present disclosure include both the surgical apparatus described above, as well as a manual morcellating trocar. The manual morcellating trocar may be placed through the wound retractor of the surgical apparatus and introduced into the specimen bag containing the tissue specimen, whereby the manual morcellator is activated to break up the tissue specimen within the specimen bag permitting removal of the tissue specimen from the body through the wound retractor. Kits of the present disclosure may also include an optional cannula for introducing the morcellating trocar into the specimen bag.

Embodiments of the present disclosure will now be described in detail with reference to the drawings, in which like reference numerals designate identical or corresponding elements in each of the several views. In the following description, well-known functions or constructions are not described in detail to avoid obscuring the present disclosure in unnecessary detail.

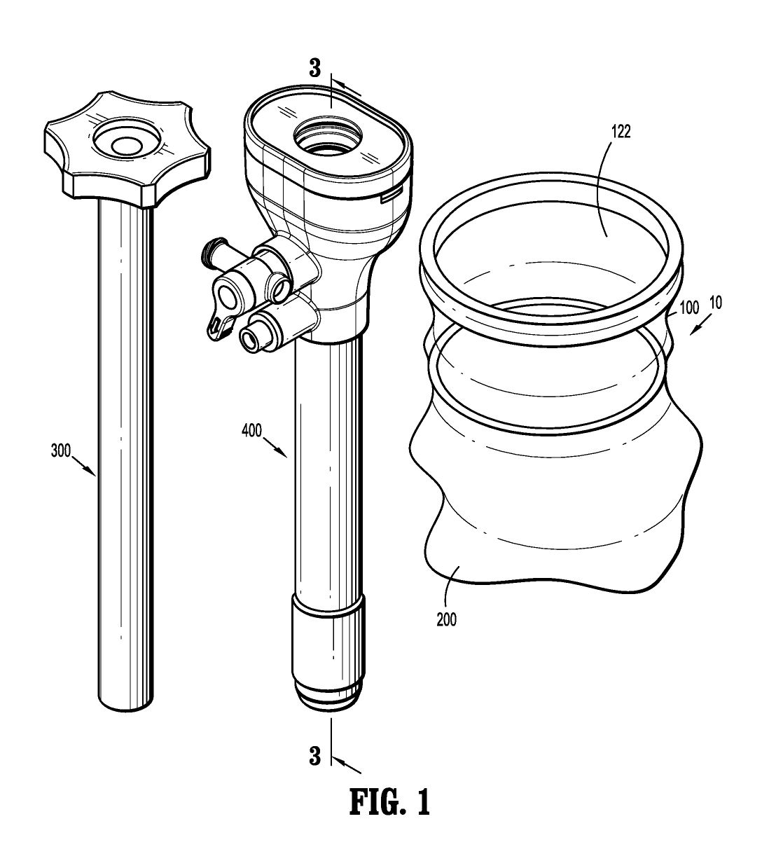

FIG. 1 illustrates a kit of the present disclosure including a surgical apparatus 10 in accordance with the present disclosure. The surgical apparatus 10 encompasses both the wound retractor 100 and the specimen bag 200. The kit of the present disclosure also includes a morcellating trocar 300 and, optionally, a cannula 400 for introducing the morcellating trocar 300 into a patient's body.

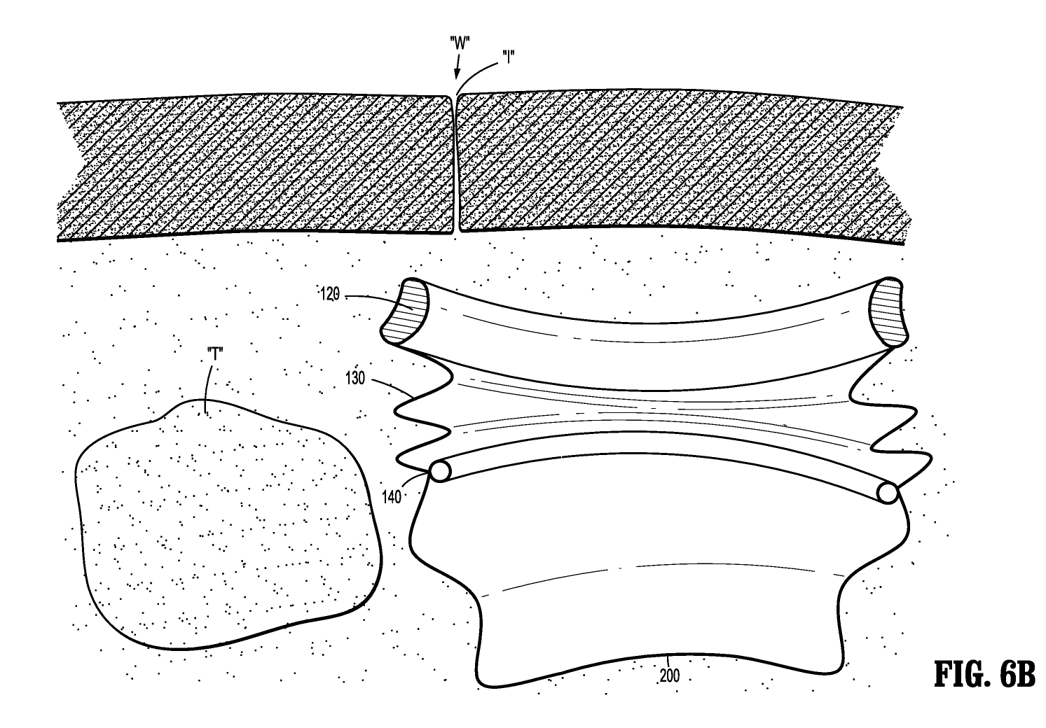

The surgical apparatus 10, including the wound retractor 100 and the specimen bag 200, is adapted for insertion into wound “W” through an incision “I” (as illustrated in FIG. 6). The incision “I” may be a single incision, e.g., through the abdominal or peritoneal lining, or a naturally occurring orifice (i.e. mouth, anus, or vagina).

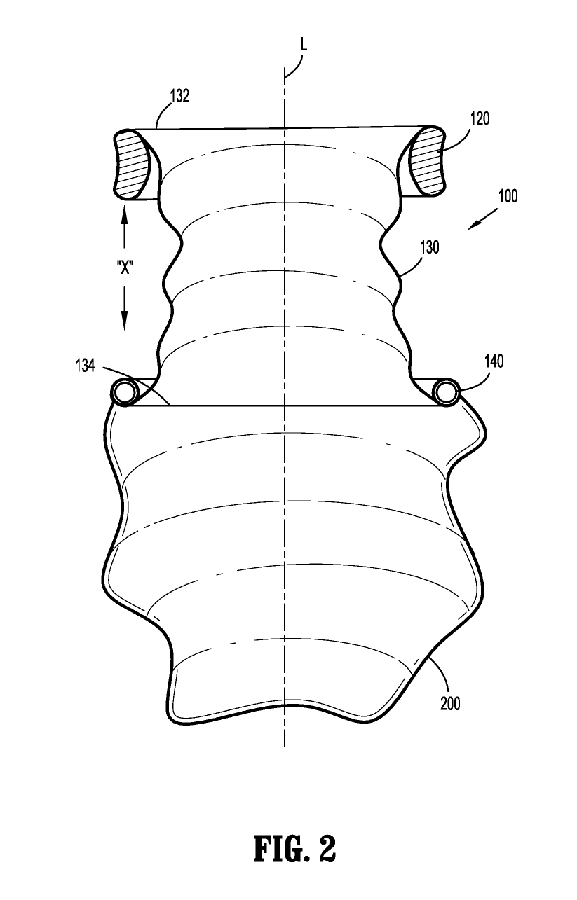

Turning to FIG. 2, the wound retractor 100 includes a proximal ring 120, a distal ring 140, and a generally cylindrical film 130 disposed therebetween. The surgical apparatus 10 further defines a longitudinal axis “L” shared by the proximal ring 120, the distal ring 140, and the film 130, as well as the specimen bag 200. It is envisioned that the proximal ring 120 and the distal ring 140 may each be detachably coupled or permanently attached to a proximal end portion 132 and a distal end portion 134, respectively, of the film 130 by any means known in the art, e.g., glue, suture, impulse welding, chemical or mechanical bonding, an over molding process, etc. In some embodiments, the proximal ring 120, film 130, and distal ring 140 are monolithically integrated such that the wound retractor 100 is a unitary structure. Attachment of the film 130 to the rings 120, 140 can be continuous around the rings 120, 140 or may be discontinuous, provided the rings 120, 140 are sufficiently secured to the film 130 to allow the film 130 to be rolled about the proximal ring 120 as described below.

The proximal ring 120 and the distal ring 140 are axially aligned along longitudinal axis “L” with the film 130 disposed therebetween, as noted above. The proximal ring 120 has a generally circular configuration that defines a circular opening 122 (see FIG. 1) and is rollable, such that the proximal ring 120 can be rolled towards or away from the distal ring 140 along the longitudinal axis “L”, which results in the film 130 being furled or unfurled about the proximal ring 120. The distal ring 140 also has a generally circular configuration that defines a generally circular opening (not shown).

It is envisioned that the proximal ring 120 and the distal ring 140 may be fabricated from resilient materials such that the proximal ring 120 and the distal ring 140 may temporarily deform into a generally oblong configuration during insertion of the wound retractor 100 through an incision “I” (FIG. 6) while reverting to a generally circular configuration during use. For example, thermoplastic polyurethanes sold under PELLETHANE®, offers flexibility and a wide range of hardnesses. The proximal ring 120, for example, may be fabricated from PELLETHANE® 2363-80A, PELLETHANE® 2363-90A, a 50/50 composition of PELLETHANE® 2363-80A and PELLETHANE® 2363-90A, or any alternatives known in the art. The distal ring 140 may be fabricated from, for example, PELLETHANE® 2363-90A for the extra small and small size, PELLETHANE® 2363-55D for the medium and large size, a 50/50 composition of PELLETHANE® 2363-90A and PELLETHANE® 2363-55D for the extra-large size, or any alternatives known in the art. The proximal ring 120 and the distal ring 140 may be fabricated from the same or different materials.

The film 130 defines a generally cylindrical shape to form a lumen between the proximal ring 120 and the distal ring 140. The circular configuration of the proximal and distal rings 120 and 140, respectively, maintains the film 130 in an expanded state to maintain the lumen in a non-collapsed state. The film 130 is coupled to the proximal ring 120 at the proximal end portion 132 and is coupled to the distal ring 140 at the distal end portion 134, such that the film 130 does not slide or move relative to a surface of either the proximal ring 120 or the distal ring 140. The film 130 may be fabricated from a clear, non-elastomeric material, e.g., a polyurethane.

The length “X” between the proximal ring 120 and the distal ring 140 is adjustable. More specifically, the length “X” can be decreased by rolling the proximal ring 120 towards the distal ring 140 to furl the film 130 about the proximal ring 120. Similarly, the length “X” can be increased by rolling the proximal ring 120 away from the distal ring 140 to unfurl the film 130 from about the proximal ring 120. As discussed above, as the proximal ring 120 is rolled towards or away from the distal ring 140, the film 130 furls or unfurls about the proximal ring 120. It should be appreciated that as the film 130 is furled about the proximal ring 120, it reduces the length “X” of the film 130, so the tension in the film between the proximal ring 120 and distal ring 140 is increased to provide a radially outward force within the incision “I”.

It is envisioned that the wound retractor 100 may come in a variety of sizes to appropriately fit and accommodate a range of incision diameters. For example, for an extra small wound retractor, the outer diameter of the proximal ring 120 and the distal ring 140 may be about 2.5 inches; for a small wound retractor, the outer diameter of the proximal ring 120 and the distal ring 140 may be about 3.9 inches; for a medium sized wound retractor, the outer diameter of the proximal ring 120 and the distal ring 140 may be about 5.1 inches; for a large wound retractor, the outer diameter of the proximal ring 120 and the distal ring 140 may be about 7.5 inches; and for an extra-large wound retractor, the outer diameter of the proximal ring 120 and the distal ring 140 may be about 9.4 inches.

Specimen bags of the present disclosure are made of flexible and durable materials within the purview of those skilled in the art, in embodiments, polymeric materials. The specimen bags are inflatable and capable of allowing a surgeon to introduce cutting devices into the specimen bag to reduce the size of the tissue specimen therein, thereby facilitating removal of the specimen bag from the body. Materials used to form the specimen bags are antistatic, pyrogen-free, non-toxic and sterilizable. In embodiments, materials used to form the film portion of the wound retractor described above may be used to form the specimen bag. In other embodiments, the specimen bag is formed of materials that are different from those used to form the film of the wound retractor. The specimen bag may be opaque or clear.

As depicted in FIG. 2, the specimen bag 200 may be joined to the distal ring 140 of the wound retractor at the distal portion 134 of the film 130 by methods within the purview of those skilled in the art, including, but not limited to, adhesive bonding, welding, heat-sealing, combinations thereof, and the like. Alternatively, the specimen bag 200 can be integrally formed with the film 130, in embodiments of the same material.

Both the proximal ring 120 and the distal ring 140 may be collapsed from the generally circular configuration to a generally oblong configuration (not shown) for insertion, along with the specimen bag 200, through incision “I”. More specifically, as the practitioner squeezes opposing sides of the proximal ring 120 and the distal ring 140 radially inwards, the generally circular openings of the proximal ring 120 and the distal ring 140 are deformed from the generally circular configuration to a generally oblong configuration (not shown) such that the proximal ring 120 and the distal ring 140 assume a smaller profile for ease of insertion through incision “I”. Once inserted, the practitioner releases the proximal ring 120 and distal ring 140 and the resiliency of the material urges the proximal ring 120 and the distal ring 140 towards their generally circular configurations.

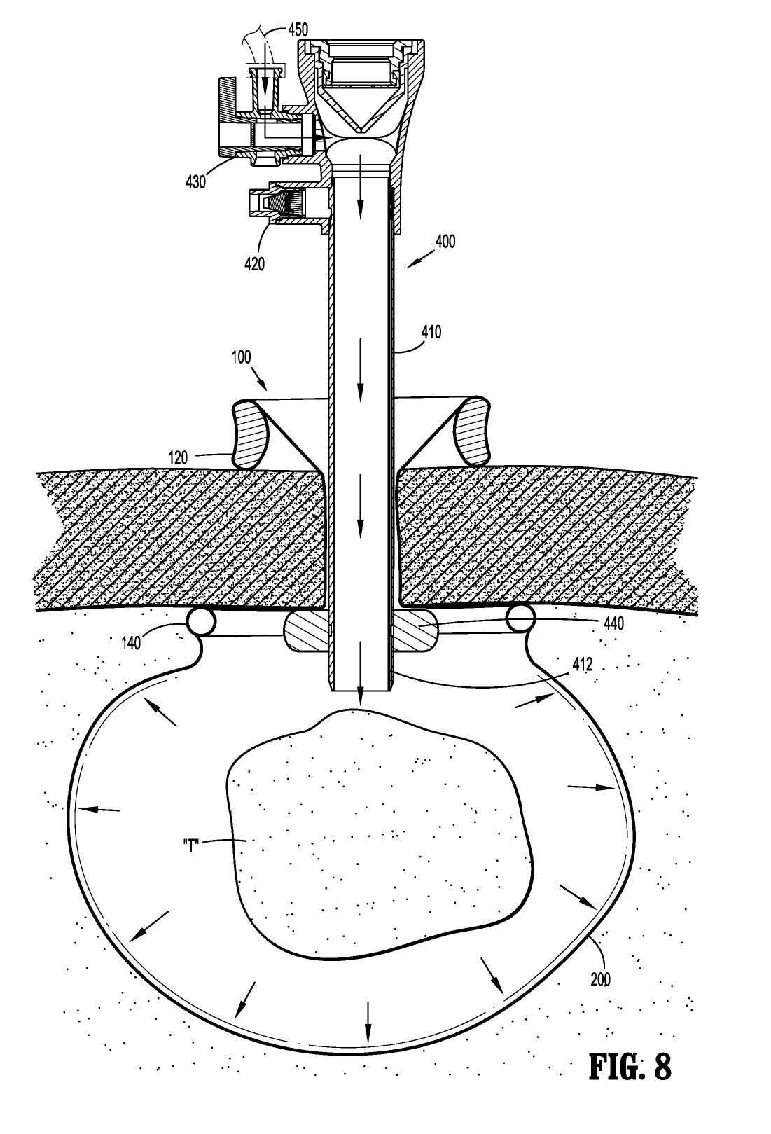

In embodiments, where the tissue specimen “T” within the specimen bag 200 is too large to pass through the wound retractor 100 (FIG. 8), a morcellator can be inserted into the specimen bag for morcellating the tissue specimen. As used herein, the term morcellator refers to a surgical instrument for cutting, mincing up, liquefying, or morcellating, tissue into smaller pieces. Care should be taken not to damage the specimen bag to prevent resected tissue from entering into a body cavity, e.g., the abdominal cavity.

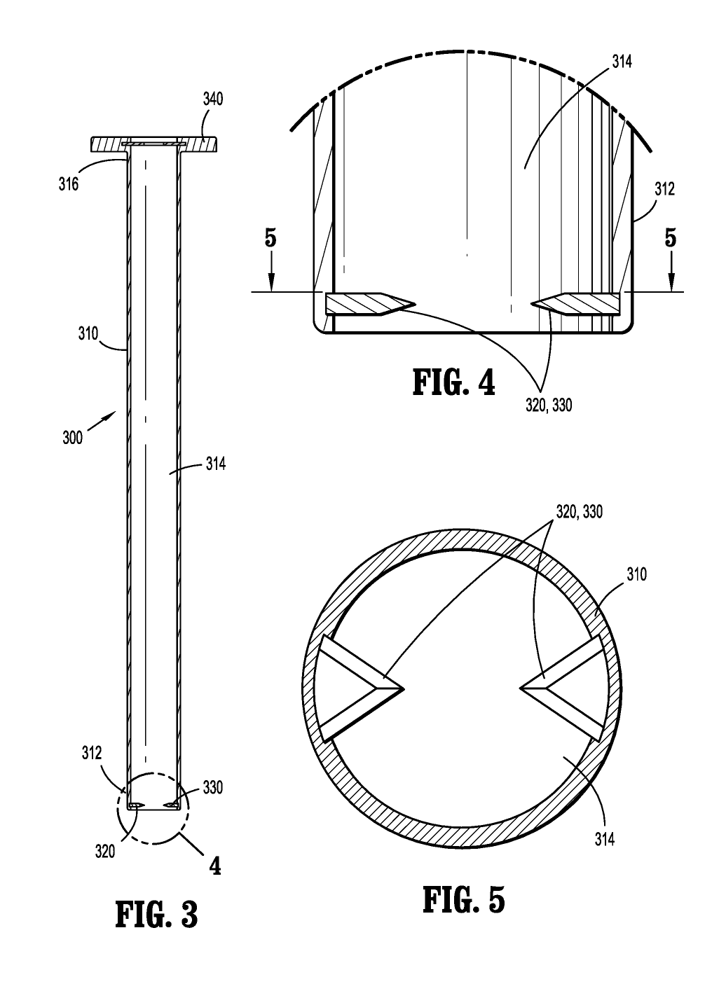

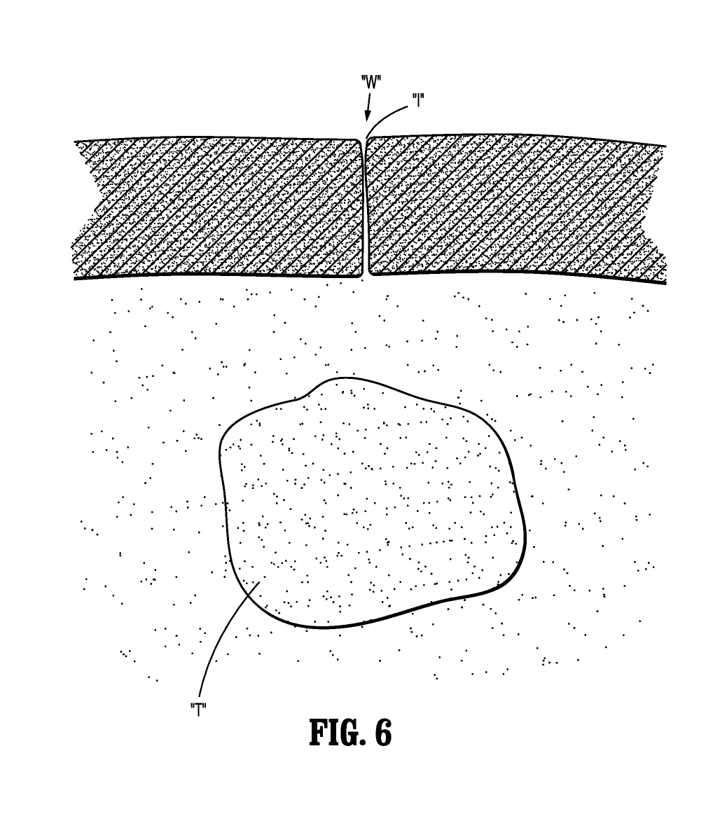

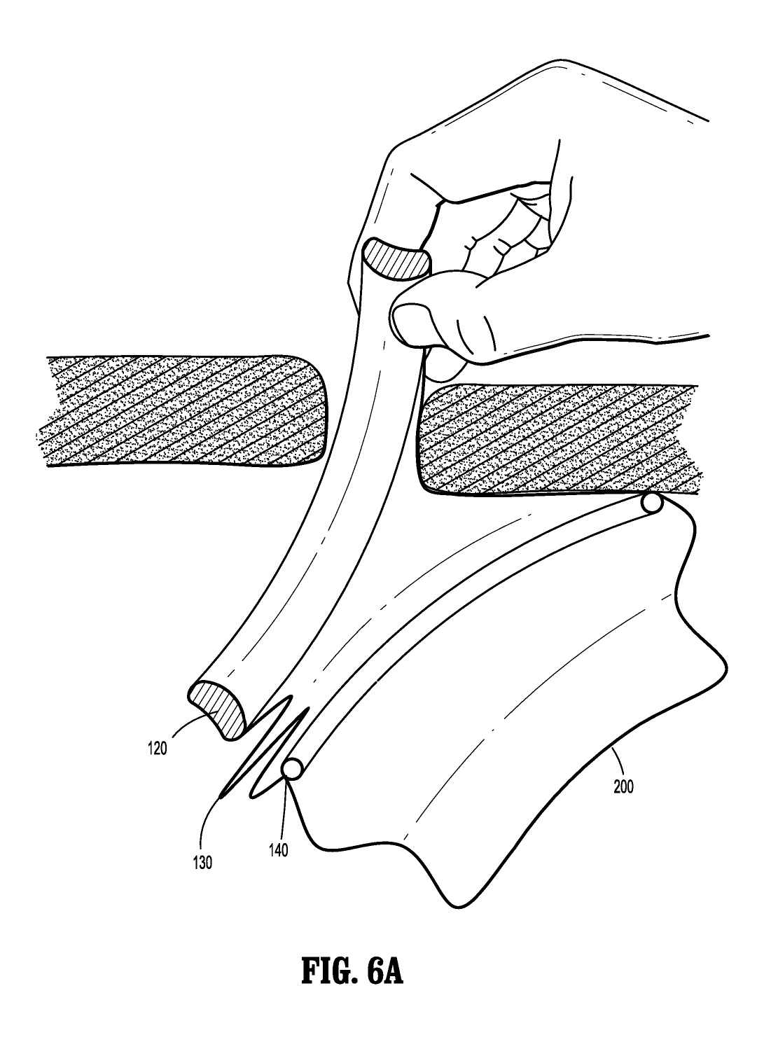

Referring to FIGS. 3-5, the kit of the present disclosure includes a morcellating trocar 300. The morcellating trocar 300 has an elongate shaft 310 defining a lumen 314. The elongate shaft 310 has a distal portion 312, with blades 320, 330 on the interior surface thereof projecting into the lumen 314 of the elongate shaft 310 along line 5-5 (FIG. 4). In embodiments, a proximal portion 316 of the elongate shaft 310 has a rotation knob 340 thereon for rotating the elongate shaft 310.

Kits of the present disclosure also include an optional cannula 400 for introducing the morcellating trocar 300 through the wound retractor 100 and into the specimen bag 200. Turning to FIG. 8, the cannula 400 has an elongate shaft 410 including inflation ports 420, 430 for introducing inflation gases into both an anchoring balloon 440 located at a distal portion 412 of the elongate shaft 410 and the specimen bag 200. The anchoring balloon 440 can be inflated to secure the distal portion 412 of the cannula 400 within the body cavity.

The anchoring balloon 440 at the distal portion 412 of the elongate shaft 410 of the cannula 400 may be inflated with any medical grade fluid, such as saline, CO₂, or any other fluid. The anchoring balloon 440 may be inflated using a syringe, mechanically or manually operated pump, or other means. The inflation port 420 for inflating the anchoring balloon 440 may be used with one-way valves, check valves, or any other valve arrangement for inflating the anchoring balloon 440.

Once the anchoring balloon 440 has been inflated to secure the distal portion 412 of the cannula 400 within a body cavity and within the specimen bag 200, the specimen bag 200 can be inflated. In order to inflate the specimen bag 200, a source of inflation pressure is releasably attached to the inflation port 430 and pressurized fluid is introduced through the inflation port 430 and communicated along axis 3-3 (as depicted in FIG. 1) of the elongate shaft 410 of the cannula 400 to the specimen bag 200.

FIG. 8 shows the cannula 400 introduced through the wound retractor 100 into the specimen bag 200 for inflating the specimen bag 200. FIG. 8 also depicts the introduction tube 450 for introducing inflation gases (arrows) into the inflation port 430 and the gases travelling from the inflation port 430 through the elongate shaft 410 into the specimen bag 200 possessing tissue specimen “T” therein.

With reference to FIGS. 6-11, a method of operating the surgical apparatus 10 in accordance with the present disclosure will be described. As shown in FIGS. 6A and 6B, the surgeon first collapses and deforms the proximal ring 120 and the distal ring 140 of the wound retractor 100 into an oblong configuration for insertion into wound “W” through incision “I”. Once the wound retractor 100 and the specimen bag 200 are placed through incision “I” and the proximal ring 120 and the distal ring 140 are released, the proximal ring 120 and distal ring 140 resume their generally circular configuration so that the surgical apparatus 10 is adjacent the tissue specimen “T” to be removed from the body cavity. The surgeon can then introduce tissue specimen “T” through the proximal ring 120, the film 130, and the distal ring 140 into the specimen bag 200 by use of a forceps, grasper, or any other suitable medical device.

Once tissue specimen “T” has been introduced into the specimen bag 200, the surgeon then collapses and deforms the proximal ring 120 using a forceps, grasper, or similar instrument. In other embodiments, the proximal ring 120 has a tether thereon (not shown), which may be pulled proximally by the surgeon to remove the proximal ring 120 from the body cavity.

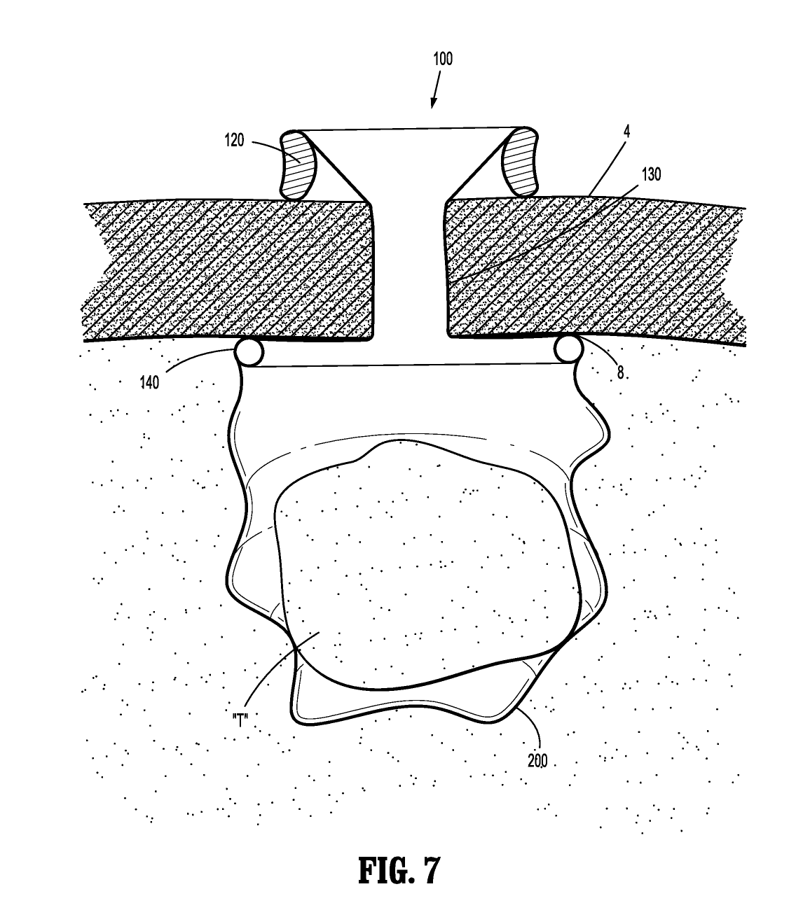

As shown in FIGS. 6A and 7, the proximal ring 120 is removed from the body cavity through incision “I” so that it rests on the outer skin 4 adjacent incision “I” and the surgeon adjusts the film 130 and the proximal ring 120, e.g., pulling the proximal ring 120 proximally to tension the film 130, such that the distal ring 140 comes into abutment with an inner surface 8 of wound “W”. The distal ring 140 is positioned adjacent the inner surface 8 of wound “W”, and reverts to the generally circular configuration so that the generally circular opening of distal ring 140 is parallel to the inner surface 8 of the wound (FIG. 7).

With the distal ring 140 in abutment with the inner surface 8 of wound “W”, the proximal ring 120 is rolled distally towards the distal ring 140 and the outer surface 4 of wound “W” to a desired position, such that the film 130 is furled about the proximal ring 120 (not shown) and thereby tensioned.

Once the wound retractor 100 and the specimen bag 200 are in position, surgical tools and instruments may pass through the wound retractor 100 with the distal ring 140 in abutment to the inner surface 8 of wound “W”, and the proximal ring 120 rolled to a desired position and/or brought into abutment to the outer surface 4 of wound “W”. The wound retractor 100 may be tensioned further to provide retraction of incision “I”, increasing the incision diameter.

Where the tissue specimen “T” in the specimen bag is too large to be removed through the incision, the morcellating trocar 300 may be introduced through the wound retractor 100 into the specimen bag 200. While the morcellating trocar 300 may be introduced directly through the wound retractor 100 into the specimen bag 200, in embodiments, as depicted in FIG. 8, the cannula 400 is first introduced through the wound retractor 100 and into the specimen bag 200. Inflation gases are introduced through the inflation port 420 on the cannula 400 to inflate the anchoring balloon 440. Once the anchoring balloon 440 has been inflated, inflation gases are introduced through the inflation port 430 on the cannula 400 to inflate the specimen bag 200.

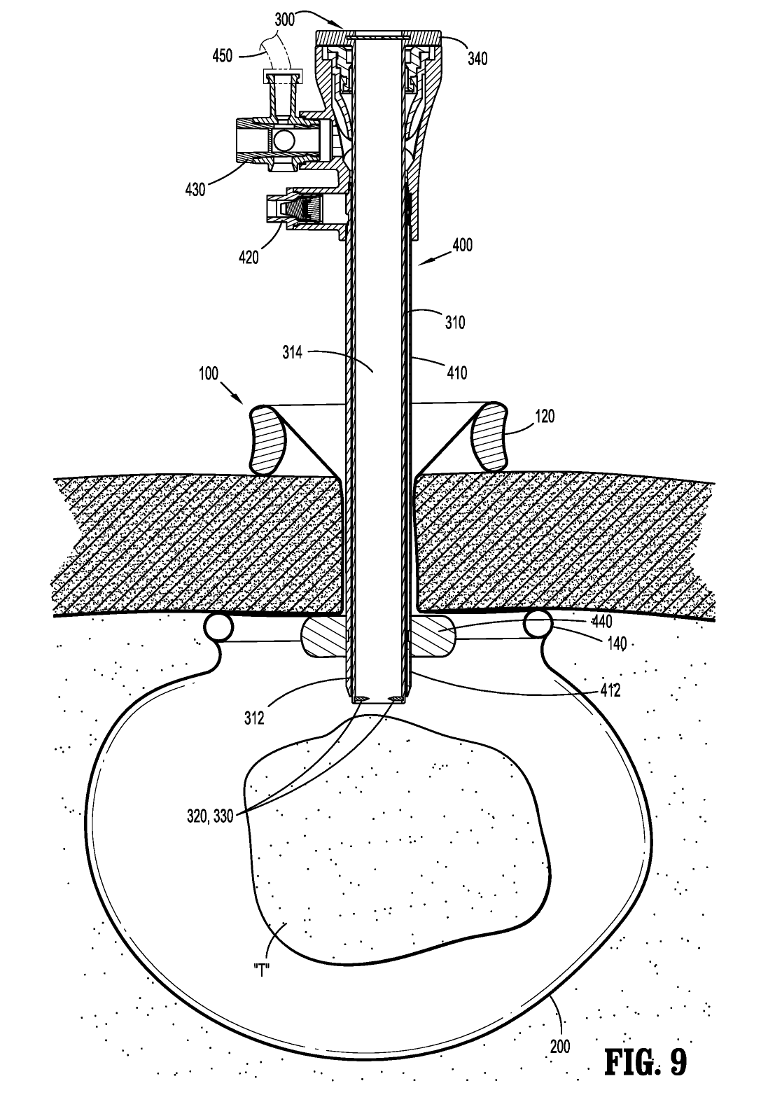

Upon inflation of the specimen bag 200, the morcellating trocar 300 is introduced through the cannula 400 into the specimen bag 200. (FIG. 9.) The distal ring 140 of the wound retractor 100 and the inflation of specimen bag 200 prevent the specimen bag 200 from coming into contact with the knife blades 320, 330 within the lumen 314 of the elongate shaft 310 of the morcellating trocar 300.

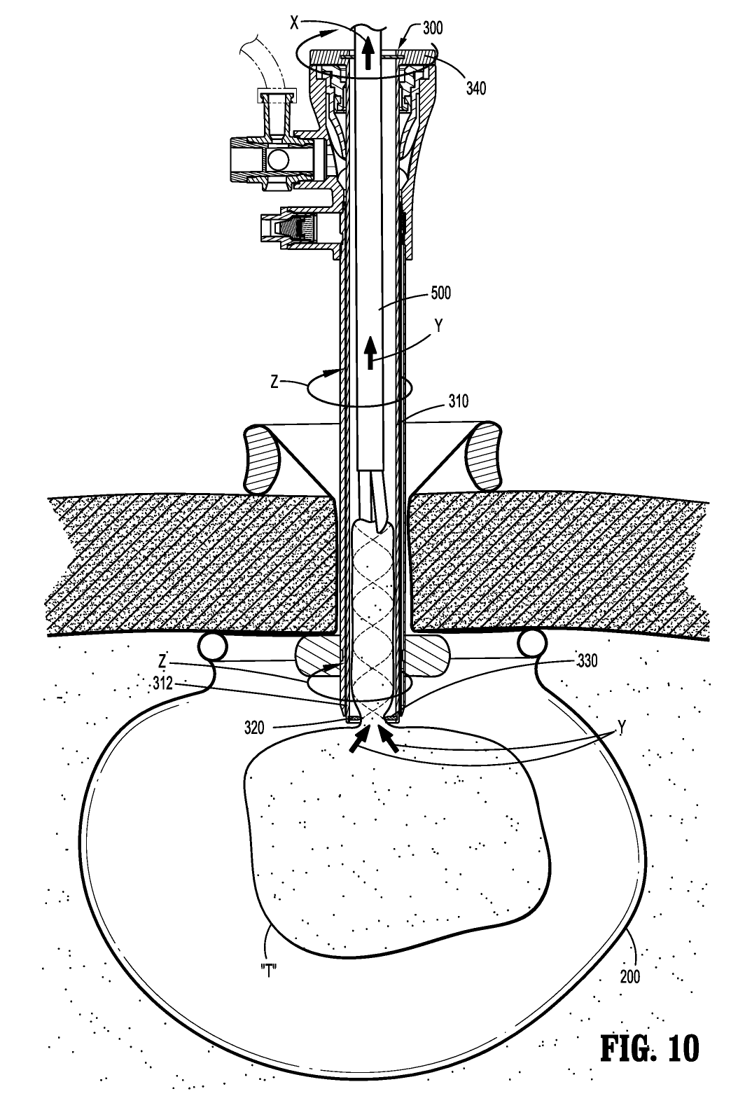

As depicted if FIG. 10, a forceps 500, or any other grasper device, may then pass through the morcellating trocar 300 into the specimen bag 200 and grasp the tissue specimen “T” therein. Although not shown, in embodiments it may be necessary to utilize a funneled introducer to constrain the forceps 500 in the center of the elongate shaft 310 of the morcellating trocar 300 so that the forceps 500 do not damage the knife blades 320, 320.

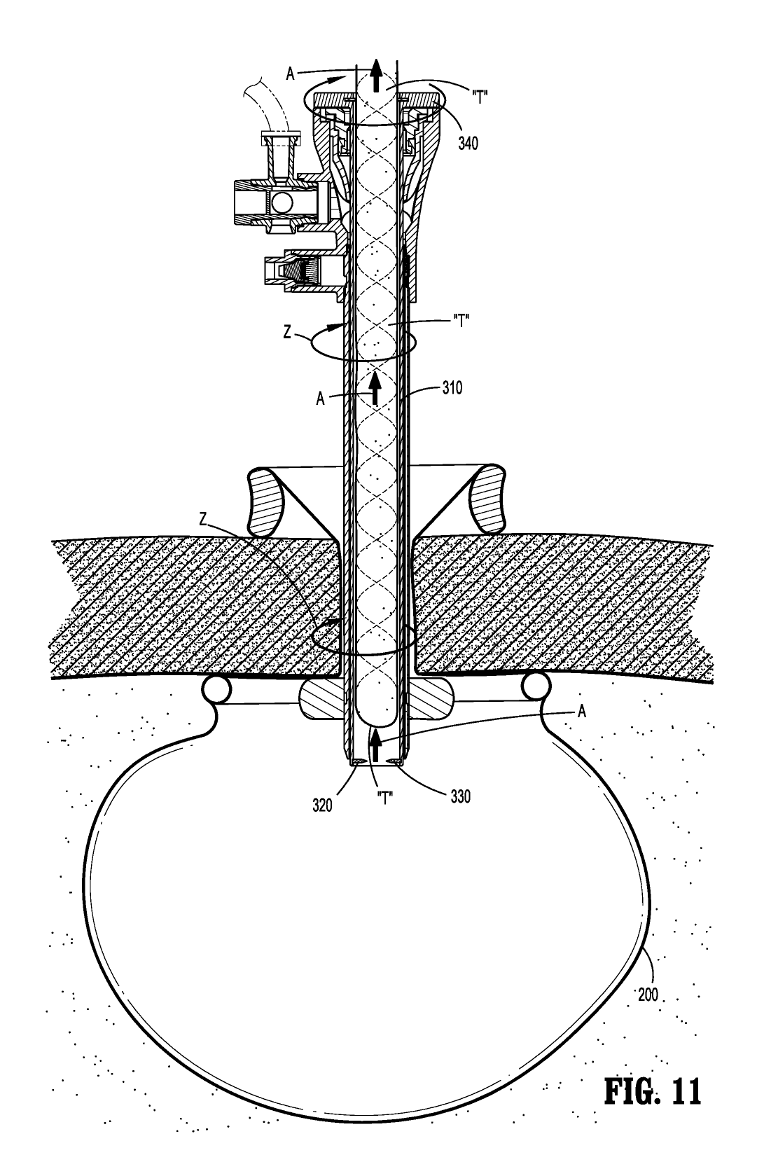

The anchoring balloon 440 on the cannula 400 helps stabilize the tissue specimen “T” during removal of the tissue specimen “T” from the specimen bag 200. As the forceps 500 are pulled proximally (indicated by arrows “Y” in FIG. 10) to remove tissue specimen “T” from the specimen bag 200, the tissue specimen “T” passes proximally through the morcellating trocar 300. As the tissue specimen “T” passes through the trocar 300, the elongate shaft 310 of the morcellating trocar 300 may be rotated within the cannula 400 as indicated by arrows “Z” in FIGS. 10 and 11 by the proximal rotation knob 340. Alternatively, or additionally, the surgeon may rotate the forceps 500 or any other grasper as the tissue specimen “T” is removed from the specimen bag 200.

Pulling tissue specimen “T” through the distal end 312 of the morcellating trocar 300 will bring tissue specimen “T” to be removed from the specimen bag 200 into contact with the blades 320, 330. Rotating the elongate shaft 310 with the proximal rotation knob 340 as tissue specimen “T” is pulled proximally into contact with the blades 320, 330 within the lumen 314 of the elongate shaft 310 of the morcellating trocar 300 will continually cut the tissue specimen “T” into a double helical pattern to allow it to fit through the smaller diameter of the elongate shaft 310. The cut tissue specimen may thus pass through the elongate shaft 310 of the morcellating trocar 300 as it is removed from the specimen bag 200. In embodiments, if tissue specimen “T” to be removed separates and portions thereof remain in the specimen bag 200 as tissue specimen “T” is cut by the blades 320, 330, the surgeon may grab those additional pieces of tissue with a forceps or other grasper, and/or use a vacuum source for removal of any remaining portion of the tissue specimen “T” from the specimen bag 200. In other embodiments, the volume of the tissue specimen “T” in the specimen bag 200, as well as any fluids from the tissue specimen “T”, may be reduced to a point that specimen bag 200, with any remaining tissue and/or fluids therein, may pass through incision “I” without need for any further morcellation.

FIG. 11 depicts the tissue specimen “T” which has been cut by the blades 320, 330 to permit removal of tissue specimen “T” through the morcellating trocar 300 (tissue travelling through the trocar is demonstrated by arrows “A”) from the specimen bag 200.

In other embodiments, not shown, power morcellators may be used with the wound retractor/specimen bag of the present disclosure.

Once a sufficient amount of tissue specimen “T” is removed from the specimen bag 200 to facilitate removal of the specimen bag 200 through incision “I”, the inflation gases are withdrawn from the specimen bag 200, and from the anchoring balloon 440. The surgeon then collapses and deforms the distal ring 140 using a forceps, grasper, or similar instrument. In other embodiments, the distal ring 140 has a tether thereon (not shown), which may be pulled proximally by the surgeon to remove the distal ring 140 from the body cavity.

The film 130, distal ring 140, and specimen bag 200 are then removed from the body cavity through incision “I”.

After the specimen bag 200 is removed, any tissue remaining therein can be removed from the specimen bag 200 for further examination or the specimen bag 200 can be discarded.

The specimen bags of the present disclosure provide safe tissue extraction at the end of minimally invasive surgical procedures. Diseased tissue may be removed from the body without seeding of spilled tissue cells inside the abdomen. The design of the wound retractor/specimen bags of the present disclosure, with the morcellating trocar described above, allows for the surgeon to break up tissue without tearing the specimen bag and possibly releasing tissue contents back into the body of the patient. It is further envisioned that the methods of using the specimen bags of the present disclosure may be modified to accommodate needs of a given procedure and/or the preferences of the surgeon. It is further envisioned that the embodiments disclosed herein may be used to remove any tissue or object from the body.

It will be understood that various modifications may be made to the embodiments disclosed herein. For example, other methods for introducing specimen bags of the present disclosure into the body of a patient may be used. Additionally, other specimen bag shapes may be used. Further, the terminology of similar components with the various embodiments should not be construed as specific to any particular embodiment. Thus, the above description should not be construed as limiting, but merely as exemplifications of preferred embodiments. Those skilled in the art will envision other modifications within the scope and spirit of the claims appended hereto.

What is claimed is:

1. A surgical apparatus, comprising:
a wound retractor including:
a proximal ring,
a distal ring, and
a film disposed between the proximal ring and the distal ring; and
a specimen bag attached to the distal ring of the wound retractor.

2. The surgical apparatus of claim 1, wherein the proximal ring of the wound retractor defines a generally circular opening and is deformable.

3. The surgical apparatus of claim 1, wherein the distal ring of the wound retractor defines a generally circular opening and is deformable.

4. The surgical apparatus of claim 1, wherein the film of the wound retractor defines an adjustable length between the proximal ring and the distal ring.

5. The surgical apparatus of claim 1, wherein the specimen bag is attached to the distal ring of the wound retractor by a method selected from the group consisting of adhesive bonding, welding, heat-sealing, and combinations thereof.

6. The surgical apparatus of claim 1, wherein the proximal ring, the distal ring, or both, possess a tether thereon.

7. A kit comprising:
a surgical apparatus including a wound retractor including:
a proximal ring,
a distal ring,
a film disposed between the proximal ring and the distal ring; and
a specimen bag attached to the distal ring of the wound retractor; and
a morcellating trocar.

8. The kit of claim 7, further including a cannula.

9. The kit of claim 7, wherein the proximal ring of the wound retractor defines a generally circular opening and is deformable.

10. The kit of claim 7, wherein the distal ring of the wound retractor defines a generally circular opening and is deformable.

11. The kit of claim 7, wherein the film of the wound retractor defines an adjustable length between the proximal ring and the distal ring.

12. The kit of claim 7, wherein the specimen bag is attached to the distal ring of the wound retractor by a method selected from the group consisting of adhesive bonding, welding, heat-sealing, and combinations thereof.

13. The kit of claim 7, wherein the proximal ring, the distal ring, or both, possess a tether thereon.

14. The kit of claim 7, wherein the morcellating trocar includes a proximal portion, a distal portion, and an elongate shaft between the proximal portion and the distal portion, the elongate shaft having blades on an interior surface at the distal portion projecting into a lumen of the elongate shaft, and the proximal portion of the elongate shaft possessing a rotation knob for rotating the elongate shaft.

15. A method comprising:
introducing into a body opening a surgical apparatus including a wound retractor having a proximal ring, a distal ring, a film disposed between the proximal ring and the distal ring, and a specimen bag attached to the distal ring of the wound retractor;
inserting the surgical apparatus through the body opening into a body cavity;
passing a tissue specimen through the proximal ring, the film and the distal ring of the surgical apparatus into the specimen bag;
removing the proximal ring from the body cavity through the body opening so that the film is adjacent tissue encompassing the body opening;
pulling the proximal ring away from the distal ring such that the distal ring is brought into abutment with an inner surface of the tissue surrounding the body opening;
rolling the proximal ring about itself such that the film is furled about the proximal ring and tensioned;
rolling the proximal ring to retract the incision and bring the proximal ring into abutment with an outer surface of the tissue; and
removing the tissue specimen from the specimen bag.

16. The method of claim 15, further including breaking up the tissue specimen prior to removing the tissue specimen from the specimen bag.

17. The method of claim 15, further including:
introducing a cannula through the proximal ring, the film, and the distal ring into the specimen bag prior to removing the tissue specimen from the specimen bag;
introducing a morcellating trocar through the cannula into the specimen bag;
introducing a grasping element through the morcellating trocar into the specimen bag and grasping the tissue specimen therein;
proximally pulling the grasping element while activating the morcellating trocar to break up the tissue specimen as it is removed from the specimen bag.

18. The method of claim 17, wherein the cannula possesses an anchoring balloon which is inflated to anchor the cannula in the body opening.

19. The method of claim 17, further comprising inflating the specimen bag prior to removal of the tissue specimen therefrom.

20. The method of claim 17, wherein the morcellating trocar includes a proximal portion, a distal portion, and an elongate shaft between the proximal portion and the distal portion, the elongate shaft having blades on an interior surface at the distal portion projecting into a lumen of the elongate shaft, and the proximal portion of the elongate shaft possessing a rotation knob for rotating the elongate shaft, and activating the morcellating trocar includes rotating the elongate shaft so that the tissue specimen is cut by the blades as the tissue specimen passes through the morcellating trocar.
